(19) United States
(12) Patent Application Publication
(10) Pub. No.: US 2010/0248372 A1
(43) Pub. Date: Sep. 30, 2010

(54) Composition and Method for Modulating Plant Transformation

(76) Inventors: Igor KOVALCHUK, Lethbridge (CA); Alex Boyko, Lethbridge (CA)

Correspondence Address: ARNOLD & PORTER LLP;ATTN: IP DOCKETING DEPT., 555 TWELFTH STREET, N.W., WASHINGTON, DC, 20004-1206, US

(21) Appl. No.: 12/411,280

(22) Filed: Mar. 25, 2009

(51) Int. Cl. C12N 5/10; C12N 5/04

(52) U.S. Cl. 435/468; 435/431

(57) ABSTRACT

A plant culture medium composition for modulating plant transformation events, comprising a plant culture medium and an effective amount of at least one compound having a chloride component intermixed thereinto. In one embodiment, the at least one chloride-containing compound is selected from the group comprising: NaCl, MgCl₂, and KCl. Another embodiment relates to a method for modulating the frequency of plant transformation events. The method comprises the steps of providing a plant culture medium composition and contacting at least one plant with the plant culture medium composition. At least one cell from the at least one plant is transformed with a nucleic acid of interest. The presence of at least one transformation event is detected and quantified. The frequency of quantified transformation events is compared with a suitable control. Changes in quantified transformations events compared to the control are indicative of changes in the frequency of plant transformation events.

### TECHNICAL FIELD

The present invention generally relates to plant growth media, particularly to plant growth medium compositions configured for modulating the frequency of plant transformation, and more particularly to plant growth media compositions having a chloride component.

### BACKGROUND ART

Agriculture is a multibillion-dollar industry that can be significantly impacted by even seemingly small improvements in methods or compositions for improving transfer of foreign genes into plants. Traditionally, methodologies based on sexual reproduction have been utilized for the transfer of genes within plant species or between closely related plant species to improve crop qualities. The pace of crop improvement by such methodologies has been slow and limited, in part due to reliance on naturally occurring gene variations in closely related species.

Advances in genetic engineering provide an alternative approach for introducing foreign genetic information into plants, thereby resulting in transgenic plants that have acquired new beneficial characteristics. Genetic engineering of plants involves genetic transformation by introducing foreign genetic material(s) in the form of a nucleic acid such as DNA, which encodes for one or more genes. Other transformation techniques, which are all well known in this field, include somatic hybridization by fusion of protoplasts and the induction of somaclonal variations in order to induce genetic modifications.

The transfer of foreign genetic material into plants is commonly performed utilizing well-known gene transfer techniques such as Agrobacterium-mediated transformation. This technique utilizes strains of Agrobacterium containing an engineered Ti plasmid to introduce the genetic material of interest. Plant tissue is cut into small pieces and soaked for about 10 minutes in an Agrobacterium suspension. These bacteria enable expression of the genetic material and produce transformants or transformed plants that exhibit profitable agronomic characteristics. Thus, it is possible to produce plants with certain desirable characteristics such as resistance to herbicides, insects, and viral diseases.

Large economic expenses have been devoted to the development of recombinant DNA technology for manipulating genetic information in plants. For example, plant genes can be cloned, and desirable genes can be recombined from unrelated organisms to confer new agriculturally useful traits to crops. Recombinant DNA technology has created a larger gene pool available for crop improvement.

However, the benefit of these advances in bioengineering can only be realized if these genes of interest can be introduced into plants reliably, consistently and economically. The increase in the efficiency of transformation rates, even by as much as two-fold, can translate into significant cost savings with respect to expenditures such as technical staff salaries, material costs and energy costs.

There are a number of methods directed to improving plant transformation efficiencies. These methods are aimed at improving the health of the bacteria that is used for transformation, the health of recipient transformed plants and the conditions during plant regeneration.

Plant transformation is by no means a routine matter. For many commercially important crop plants, the efficiency or frequency of transformation is calculated by dividing the number of transformed plants produced by each transformation attempt. Both the efficiency and frequency is very low and highly variable among genetic lines and varieties. Some highly desirable breeding lines exhibit extremely low transformation frequencies relative to other genetic lines of the same crop species. In some cases, satisfactory levels of transformed plant cells and calli can be achieved from a transformation attempt, but such transformed cells and calli are resistant to regeneration into transformed embryos and plants.

The prior art methods generally result in poor control over where and how the DNA of interest is integrated into the plant genomic DNA during transformation. The introduced genetic material typically integrates randomly and is mediated primarily via non-homologous end-joining thus leading to frequent inactivity of the transgene and/or modification of the genomic sequences due to integration of truncated copies of the DNA, multiple integrations, and deletions at the site of integration. Also, the prior art methods are only aimed at improving one of the transformation steps in gene transfer.

It is known that double strand breaks are associated with transformation so that the foreign DNA of interest can integrate into the plant genomic DNA. Repair of the DNA strand breaks are mediated by two major mechanisms or pathways, namely non-homologous end-joining and homologous recombination. Researchers have revealed that non-homologous end-joining is an error-prone mechanism and frequently results in deletions and/or insertions at the place of the repair where the integration has occurred. In contrast, homologous recombination is considered error-free and, therefore, a more desirable mechanism for DNA integration during plant transformation. However, non-homologous end-joining is the predominant repair mechanism in plants. It has been shown that the ratio of non-homologous end-joining to homologous recombination is at least about 1000:1 in plants.

The inability to control where and how genes are integrated and the errors introduced during transformation are major drawbacks of existing methodologies in gene transformation. It is currently unclear what factors control the preferential utilization of non-homologous end-joining over homologous recombination for the repair of double strand breaks in plants. Both of these mechanisms play a role in the integration of foreign DNA with respect to transformation. Given that non-homologous end-joining is the predominant mechanism utilized in plants, an increase in homologous recombination can lead to more effective integration of the desired gene, more intact “clean” integration and greater control in targeting genes to their desired locations.

### SUMMARY OF THE INVENTION

Exemplary embodiments of the present invention are related to plant culture medium compositions and methods for modulating the frequency of plant transformation events.

One exemplary embodiment of the present invention relates to a plant culture medium composition configured for modulating plant transformation events. The composition comprises a plant culture medium and an effective amount of at least one compound having a chloride component intermixed thereinto. According to one aspect, a baseline level for plant transformation events is provided by culturing at least one of a plurality of plant cells, at least one plant, and plant tissue on the plant culture medium.

In a further exemplary embodiment, on comparison to the baseline level for plant transformation events, the plant culture medium composition increases the number of plant transformation events. According to one aspect, on comparison to the baseline level for plant transformation events, the plant culture medium composition increases the number of plant transformation events by at least one of 2-fold, 3-fold, 4-fold, 5-fold, and 10-fold.

A further exemplary embodiment of the present invention relates to a method for modulating the frequency of plant transformation events. The method comprises the steps of providing a plant culture medium composition where the composition comprises a plant culture medium and an effective amount of at least one compound having a chloride component intermixed thereinto. At least one plant is then contacted with the plant culture medium composition, and at least one cell from the at least one plant is transformed with a nucleic acid of interest. The presence of at least one transformation event is then detected and the transformation events quantified. The frequency of the quantified transformation events is then compared with a suitable control. Changes in the quantified transformations events compared to the control are indicative of a change in the frequency of plant transformation events. According to one aspect, changes in the quantified transformations compared to the control, are an increase in the frequency of plant transformation events. According to another aspect, changes in the quantified transformations compared to the control are an increase in the frequency of plant transformation events by at least one of 2-fold, 3-fold, 4-fold, 5-fold, and 10-fold.

In another exemplary embodiment, the at least one chloride-containing compound is selected from the group comprising: NaCl, MgCl₂, and KCl. According to a one aspect, the chloride containing compound is KCl. According to yet another aspect, KCl is provided in an amount of at least 47 mM. According to a further aspect, KCl is provided in an amount greater than at least 18.8 mM.

In one exemplary embodiment, a suitable control is selected from the group comprising a stored dataset of results generated from studies of the presence and expression transformation events in one or more population(s) of plants grown on the plant culture medium, a stored dataset of results generated from studies of the presence and expression of transformation events in one or more population(s) of plant cells grown on the plant culture medium, a stored dataset of results generated from studies of the presence and expression transformation events in one or more population(s) of plant tissue grown on the plant culture medium and combinations thereof.

Another exemplary embodiment of the present invention relates to a method for transforming a plant cell. The method comprises the steps of providing a plant culture medium composition where the composition comprises a plant culture medium and an effective amount of at least one compound having a chloride component intermixed thereinto. A plurality of plant cells are contacted with the plant culture medium composition and the plurality of plant cells are transformed with a selected nucleic acid. The presence of at least one transformation event is detected and at least one transformed plant is regenerated from at least one transformed plant cell.

Further aspects of the invention will become apparent from consideration of the ensuing description of preferred embodiments of the invention. A person skilled in the art will realize that other embodiments of the invention are possible and that the details of the invention can be modified in a number of respects, all without departing from the inventive concept. Thus, the following drawings, descriptions and examples are to be regarded as illustrative in nature and not restrictive.

### BRIEF DESCRIPTION OF THE DRAWINGS

Embodiments of the present invention will now be described, by way of example only, with reference to the attached Figures, wherein:

FIG. 1 is a block diagram showing the structure of a gene construct useful for plant transformation with an exemplary embodiment of the present invention;

FIG. 2(a) is a block diagram showing homologous recombination events with the GUS marker gene, and FIG. 2(b) is a companion image showing plants transformed with the GUS-marker gene;

FIG. 3 is a chart showing the effects of different ion combinations on homologous recombination frequency;

FIG. 4 is an image showing the effects of increasing concentrations of NaCl on the development of biomass by Arabidopsis;

FIG. 5 is a chart showing the effects of increasing concentrations of KCL on the homologous recombination frequency in Arabidopsis;

FIG. 6 is a chart showing the effects of increasing concentrations of KCL on the numbers of calli regenerated by Nicotiana tabacum; and

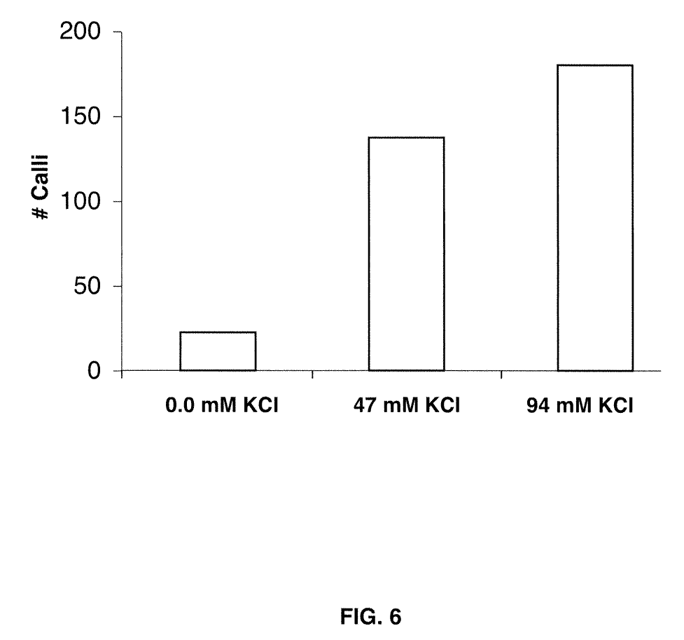

FIG. 7 is a chart showing the effects of increasing concentrations of KCL on the regeneration of stable N. tabacum transformants.

### DETAILED DESCRIPTION OF THE INVENTION

Embodiments of the present invention relate to methods and compositions configured for modulating plant transformation, more particularly for increasing plant transformation frequency. Some embodiments relate to plant culture medium compositions for modulating plant transformation, specifically homologous recombination. The present invention further relates to methods for transforming plants and methods for modulating the frequencies of plant transformation. It was discovered by the present invention that growing plants on medium enriched with chloride containing compounds, in particular potassium chloride (KCl), affects increases in the homologous recombination rates of plants without causing physiological damage to the plants.

Homologous recombination is a type of genetic recombination, a process of physical rearrangement occurring between two strands of DNA. Homologous recombination involves the alignment of similar sequences, formation of a Holliday junction, and breaking and repair, known as resolution, of the DNA to produce an exchange of material between the strands. The process of homologous recombination naturally occurs in plants. Homologous recombination is the mechanism of crossing-over in meiosis, and this mechanism creates diversity in the plant population. Breeders rely on this diversity when breeding new plant varieties. Thus, growing plants on a medium that is known to augment the rate of homologous recombination may also allow for higher diversity in the plant progeny grown on that same medium.

The present invention also relates to the addition of chlorides, particularly KCL, in specific concentration ranges for increasing the frequency of homologous recombination.

There are several technologies known in the art that may be used to transform plant cells with selected DNA molecules. These technologies are well known to those persons skilled in the art and may include, but are not limited to: (1) chemical methods; (2) physical methods such as microinjection, electroporation, and particle bombardment; (3) viral vectors; (4) receptor-mediated mechanisms; and (5) Agrobacterium-mediated plant transformation methods. Further methods may be used to accelerate DNA-coated metal particles into living cells including, but not limited to, pneumatic devices; instruments utilizing a mechanical impulse or macroprojectile; centripetal, magnetic or electrostatic forces; spray or vaccination guns; and apparatus based on acceleration by shock waves, such as electric discharge.

Further, in selecting the appropriate method for transforming cells there are additional variables or parameters that may be considered and tested, which are known to those skilled in the art. These may include physical parameters such as: (1) the nature, chemical, and physical properties of the metal particles; (2) the nature, preparation, and binding of the DNA onto the particles; and (3) the characteristics of the target plant tissue. These may also include environmental variables such as temperature, photoperiod and humidity of donor plants, explants, and bombarded tissues as well as biological factors.

In one exemplary embodiment, Agrobacterium-mediated transformation may be used for transforming plants, more specifically crop plants such as monocots and dicots exemplified by Nicotiana tabacum (tobacco), Brassica spp. (canola), Solanum tuberosum (potato), Solanum lycopersicum (tomato), Zea mays (maize), Triticum spp. (wheat), Oryza sativa (rice), Papevar spp. (poppy), and xTriticosecale (triticale). There are several Agrobacterium species that are known in the art, which can mediate the transfer of the DNA, known as “T-DNA”. T-DNA may be genetically engineered to carry a specific piece of DNA of interest into selected plant types or species. Some major events marking successful transformation can include, but are not limited to, induction of virulence genes, processing and transfer of the T-DNA to the plant's genome.

Typically, prior to actual transformation, the nucleic acids or genetic components of interest for introduction into plant cells or tissues are selected. Genetic components can include any nucleic acid that is capable of being introduced into a plant cell or tissue. The genetic components can include non-plant DNA, plant DNA, or synthetic DNA. In an exemplary embodiment, the genetic components of interest are incorporated into a DNA composition such as a recombinant, double-stranded DNA construct in the form of a plasmid or vector molecule. DNA constructs in the form of plasmids or vectors typically consist of a number of genetic components, including but not limited to regulatory elements such as promoters, leaders, introns, and terminator sequences. The DNA construct may further comprise a number of genetic components to facilitate transformation of the plant cell or tissue and regulate expression of the desired gene(s). Method for preparation of DNA constructs in the form of plasmids or vectors containing the desired genetic components are well known in the art.

Promoters used in DNA constructs, which are active in plant cells are known in the art. These promoters may include, but are not limited to, 35S, 1′/2′, actin, tubulin, and chalcone synthase promoters. Such promoters can be used to create various types of DNA constructs for expression in plants. Promoter hybrids can also be constructed to enhance transcriptional activity or to combine desired transcriptional activity, inducibility, and tissue or developmental specificity.

Genes or DNA of interest for use as a selectable, screenable, or scorable marker are exemplified by beta-glucuronidase (GUS), green fluorescent protein (GFP), luciferase (LUC), antibiotics like kanamycin and hygromycin, and herbicides like glyphosate. Other selection devices can also be implemented, including, but not limited to, tolerance to phosphinothricin, bialaphos, and positive selection mechanisms.

Any suitable plant transformation plasmid or vector can be used in the present invention with the methods disclosed herein. The plasmid construct may contain a selectable or screenable marker and associated regulatory elements as described above, along with one or more nucleic acids, for example a structural gene or DNA of interest, expressed in a manner sufficient to confer a particular desirable trait into selected plant cells. Examples of suitable structural genes may include, but are not limited to, genes selected for modulating plant tolerance to insect and/or microbial pests, genes selected for modulating plant tolerance to herbicides, genes selected for conferring quality improvements to target plant cells such as yield increases, nutritional enhancements, increased tolerances to environmental and/or physiological stresses, or genes suitable for modulating any desirable changes in plant physiology, growth, development, morphology, or plant product(s).

One exemplary embodiment relates to a plant growth medium composition for modulation of plant transformation events. The composition contains a plant culture medium suitable for growing plants, into which an effective amount of at least one chloride-containing compound is provided. Intermixing of the at least one chloride-containing compound with the plant culture medium provides a composition for increasing plant transformation frequency. A baseline level for plant transformation events is provided by culturing at least one plant or plurality of plant cells on the plant culture medium which does not contain the chloride containing compound.

The chloride-containing compound may be selected from the group comprising NaCl, MgCl₂, and KCl.

In a further embodiment, the plant growth media composition may additionally include compounds for further increasing the frequency of plant transformation events. These compounds are exemplified by rare earth element-containing compounds, nitrate-containing compounds, and combinations thereof.

The term “plant growth medium” as used herein, refers to the plant growth culture media, in any of liquid, solid, or semisolid form used before, during, or after the transformation of the plant cells, tissues, parts, or other plant tissue explants and subsequent regeneration of whole, transgenic plants therefrom. Depending upon the plant species being transformed and the transformation process being used, the media may comprise one or more of isolation media, preculture media, induction media, inoculation media, delay media, selection media, or regeneration media. The plant cells or tissues may include, but are not limited to, immature embryos, scutellar tissue, suspension cell cultures, immature inflorescence, shoot apical meristem, nodal explants, callus tissue, hypocotyl tissue, cotyledons, roots, and leaves.

Another exemplary embodiment relates to a method for modulating the frequency of plant transformation events. The method comprises the steps of providing a plant culture medium composition suitable for growing plants comprising a plant culture medium and an effective amount of at least one chloride-containing compound. Then, at least one plant is contacted with the plant culture medium composition. A plurality of plant cells from the plant are then transformed with a nucleic acid of interest, after which, the plant cells are cultured and subsequently assessed for the presence of transformation events. If detected, the transformation events are quantified. The frequency of the quantified transformation events is then compared with a suitable control. Changes in the quantified transformation events compared to the control are indicative of a change in the frequency of plant transformation events.

### Methods and Materials

Preparation of DNA Constructs:

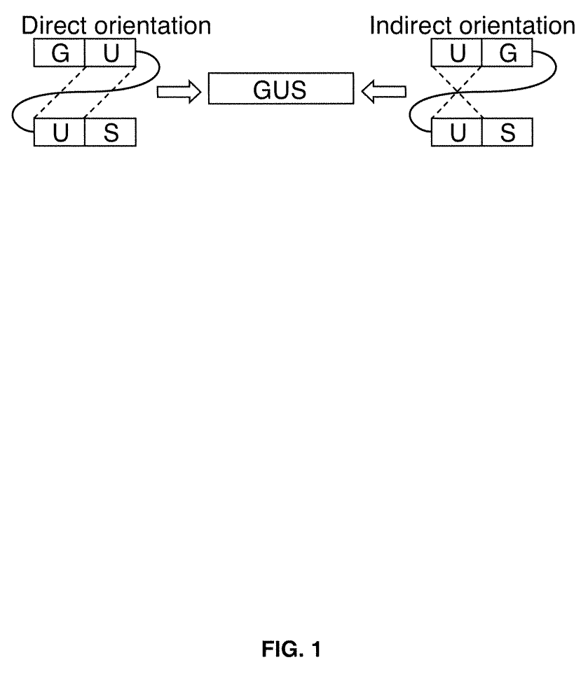

DNA constructs were prepared using gene integration according to standard molecular biology techniques, known to those skilled in the art. FIG. 1 illustrates exemplary structural arrangements of DNA constructs containing the reporter markers LUC and hygromycin.

Agrobacterium tumefaciens strain GV3101, otherwise known as AtvirD2, (Tinland et al., EMBO J., 1995, 14(14): 3585-3595) carrying a selectable marker encoding for a gene product for conferring resistance to the antibiotic rifampycin, was transformed with DNA construct comprising the LUC and hygromycin genes. The LUC and hygromycin genes were cloned in between two T-DNA borders, the left border (LB) and the right border (RB) allowing the processing by the Agrobacterium cells and delivery of the entire T-DNA portion. The Agrobacterium cells contained the screenable, or scorable marker gene encoding for the LUC gene. The LUC marker was used for quantifying transformation events. Hygromycin enabled selection of the transformants that were resistant to the antibiotic hygromycin.

Transformed Agrobacterium cells were selected by culturing the transformed cells in a medium containing 50 μg/ml of spectinomycin. The spectinomyin-resistant Agrobacterium cells were then harvested, re-plated onto fresh spectinomycin-containing media, and the resulting colonies were used to inoculate a 4-mL liquid culture containing YEB medium supplemented with 10 mM magnesium sulfate, 100 μg/mL of ryphampycin, and 50 μg/mL of hygromycin.

The liquid culture was incubated overnight and 500 μL of the Agrobacterium culture was used to prepare a 100-mL culture. Agrobacterium cultures having optical densities in a selected range of about 1.5 to about 2.0 were collected, and washed with 10 mM of magnesium sulfate. A pellet was obtained by centrifugation after the washing step, and was re-suspended in 50 mL of MS medium having a pH of about 5.2. This Agrobacterium suspension was vacuum infiltrated.

Detection of Homologous Recombination:

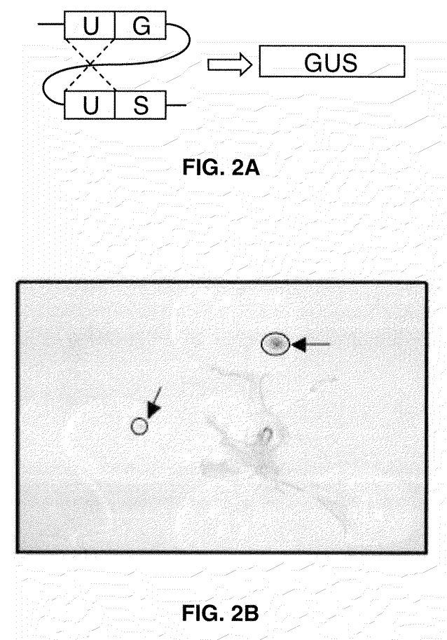

Homologous recombination was detected in plants, in particular Arabidopsis thaliana and tobacco plants, that were transformed with scorable reporter markers, for example, beta-glucuronidase (uidA or GUS). Upon homologous recombination, the marker gene is restored. Homologous recombination events were identified using histochemical staining. An exemplary imaging result is shown in FIG. 2, where the sites of homologous recombination events on transformed plants are visualized as brightened blue regions following histochemical staining and subsequent washing with ethanol. A recombination substrate generally consists of two non-functional overlapping copies of a GUS gene. Damage to one of the regions of homology may be repaired using the second copy as a template. A simple count of the number of recombination events in a population of plants was used to conduct quantitative analyses of beta-glucuronidase (GUS) activity. The homologous recombination frequency (HRF) was determined by relating the number of blue spots counted which are indicative of transformation events and then relating that number to the total number of plants scored. The recombination rate (RR) was determined by relating the HRF to the total number of haploid genomes present in the plant.

Counting of Regenerated Transformation Events:

The number of transgenic plants, having incorporated a marker gene, regenerated from tobacco calli in various transformation experiments were counted. All the regenerated plants were screened using a luciferase camera. Spraying the transgenic plants with luciferine, the substrate for luciferase enzyme, allowed the identification of transgenic plants expressing the luciferase.

Calculation of Genomic Number in Plants:

The total DNA of the transgenic lines was isolated from whole plants at preferably the full rosette stage using a Nucleon™ PhytoPure™ plant DNA extraction kit from Amersham Life Science. DNA may also be isolated from plants at a different development stage. The yield of total DNA measured in one of micrograms per plant or micrograms per plant organ was compared with the known mean DNA content, 0.16 pg of an A. thaliana cell, to give an approximate number of genomes present in plants (Swoboda et al., Mol. Gen. Genet., 1993, 237(1-2): 33-40). The total DNA was isolated from one of all leaves, roots, and stems of 4 plants per each experimental group for each transgenic line. The average DNA content from these samples was used to estimate the number of genomes present.

For calculation of the approximate number of genomes present in lateral and medial parts of the leaves, the leaves were cut into two halves. Twelve groups of 8 leaves each were prepared and DNA content measured. The total amount of DNA measured from the lateral or medial part of the 8 leaves was divided by number of leaves used to get an average DNA content per leaf. The DNA content was also measured for nine groups of plants sampled at the age of 2, 3, 5, 7, 10, 13, 16, 19 and 22 days post-germination, where between about 4-60 plants were present in each group.

To determine whether the DNA extraction method had a significant influence on the DNA yield, the DNA was isolated and content measured using an alternate protocol. The tissue from four 3-week-old Arabidopsis plants were snap frozen, grinded, and homogenized in 400 uL of an extraction buffer (200 mM Tris-Cl pH 5; 250 mM NaCl; 25 mM EDTA; 0.5% SDS), and transferred to 1.5 mL Eppendorff tubes. After the addition of 6 uL of 2-mercaptoethanol, the tubes were vortexed and stored at about 65° C. for a period of 30 to 45 minutes with occasional vortexing. The tubes were then centrifuged for a period of about 5 minutes at 3300 rpm, after which the supernatant was collected and transferred to new tubes. An equal volume of phenol was added to each of the tubes and the tubes were then mixed vigorously for a period of about 20 to 30 seconds. After centrifugation at a maximum speed 12,000 rpm for a period of about 2 minutes, the aqueous upper phase material was then collected and transferred to new tubes. An equal volume of chloroform was added to each of the tubes and the contents were well mixed. Tubes were then centrifuged at a maximum speed of 12,000 rpm for a period of about 2 minutes. The upper aqueous phase material was again transferred to new tubes and RNAase was added to a final concentration of 20 ug/mL. The tubes were then incubated for about 30 minutes at 37° C., and a 1/10 volume of 3M sodium acetate, pH 5.0 and 1 volume of cold isopropanol were added to each tube. The tubes were stored for about 30 minutes at −20° C. and then centrifuged at a maximum speed of 12,000 rpm for about 15 minutes. Pellets of material collected from the tubes were washed with 1 mL of cold, 70% ethanol, centrifuged at a maximum speed of 12,000 rpm for a period of about 5 minutes, and then dried and re-suspended in sterile distilled de-ionized water. DNA contents were then measured on a spectrophotometer.

While the DNA yields were somewhat different between the two methods used, the ratio between the amounts of DNA in plants grown at different conditions was the same. For the experiments detailed below, the Nucleon™ PhytoPure™ plant DNA extraction method was used.

Bacterial Culture:

The Agrobacterium cultures were streaked on plates containing solid YEP medium supplemented with a suitable antibiotic, for example hygromycin. The plates were incubated at 28° C. overnight. A single colony was then used to start a small 3 ml liquid culture of YEP supplemented with antibiotics. The 3-ml bacteria culture was incubated overnight at 28° C. in a rotary incubator between about 190-200 rpm. The 3-ml liquid culture was used to inoculate a primary 150 ml culture that was then grown overnight under the same conditions. Following incubation, bacteria were harvested (5000 rpm, 5 min) and re-suspended in ½-strength MS medium to a final optical density of 0.6 measured at 600 nm. The resultant bacterial suspension was then supplemented with a 100 mM acetosyringone solution to a final concentration of 100 uM. The bacterial suspension was then incubated for at least 30 minutes to stimulate the bacteria. Following incubation with acetosyringone, the bacteria were used for transformation.

Plant Growth Conditions:

Seeds of tobacco cultivar “Big Havana” were surface-sterilized with a solution containing 1% bleach and 0.05% Tween-80, for about 3 minutes and then rinsed twice with sterile distilled water for about 5 minutes. Surface-sterilized seeds were plated in 100 mm Petri dishes on sterile Whatman® filter paper submersed in 4 ml of liquid MS medium containing varying amounts of KCl and the plants were transferred to a growth chamber for germination. Once germinated, the plants were removed from the growth chamber and grown for a period of one week under conditions of 16-hours light, 22° C. and 8-hours dark, 18° C. Three to five one-week-old plants were then removed from the 4-ml liquid medium and were transferred to single sterile glass 250-ml flasks containing 15 ml of sterile liquid MS media supplemented with varying amounts of KCl. Flasks were then installed on shakers at 50-75 rpm. Plants were continuously grown under conditions of 16-hours light, 22° C. and 8-hours dark, 18° C. The growth medium in each flask was replaced weekly with 25 ml of fresh medium. Following a 3-week period, plants were removed from the flasks and 2 to 3 pairs of fully developed fresh leaves about 2-4 cm long were harvested (cut from the plant) for transformation with Agrobacteria.

Plant Growth Media:

Murashige Skoog (“MS”) medium was used as the base plant growth medium. Standard MS medium generally contains 20.6 mM of ammonium nitrate and 18.8 mM of potassium chloride. Other plant growth media known to those skilled in the art may also be used, such as the Gamborg's B5 medium or Chu's N6 medium.

Plant Transformation:

Experimental groups of tobacco plants were germinated and grown in a liquid medium culture supplemented with varying amounts of KCl. Control plants for the transformation experiments were grown in a standard MS-medium that was not supplemented with KCl.

Four weeks post-germination, the tobacco plants were removed from the liquid medium culture. The leaves from the plants were removed, and several parallel incisions were made along the leaves. The leaves were then vacuum infiltrated with an Agrobacterium suspension culture carrying the plasmid with LUC (gene coding for luciferase) and hygromycin (gene coding for the resistance to hygromycin) genes.

The leaves were vacuum-infiltrated twice for about 5 minutes with the Agrobacterium suspension culture using standard procedures known in the art. Following vacuum-infiltration, the tobacco leaves were dried for about 5 to 10 minutes on sterile Whatman® filter paper to remove substantially all excess Agrobacterium cells. The leaves were then placed on plates containing MS medium, and each of the plates were placed for in a room for a period of 3 day at a temperature of 22° C. and exposed to a daily regime of 16-hours of light and 8-hours of dark.

Leaves from each of the experimental groups grown on the different media compositions having varying amounts of KCL and the controls were washed with sterile water to remove the Agrobacterium suspension. To remove traces of growth medium, leaves were blotted on sterile filter paper and then submersed in a Petri dish laid out with Whatman® filter paper containing re-suspended Agrobacterium cells. Each submersed leaf surface was incised using a sharp surgical blade in parallel along the side veins. The distance between the two parallel incisions was about 5-7 mm. The primary leaf vein and leaf margins were left intact. Once cutting was complete, leaves remained submersed for a period of about 10 minutes. Leaves were then removed from the Petri dish and were blotted dry and placed upside-down on plates of MS medium, and were incubated in a dark room at 22° C. for a period of 3 days. Following incubation, leaves were removed from the plates and rinsed with sterile distilled water, blotted dry and transferred onto solid MS medium containing 0.8 mg/L of indole-3-acetic acid (IAA), and 2 mg/L kinetin for calli induction and regeneration, a combination of 100 mg/L ticarcillin and 3 mg/L potassium clavulanate to control Agrobacterium growth, and 25 mg/L hygromycin for selection for transformed cells.

After a period of about 3 to 4 weeks, the numbers of regenerated calli were determined. Shoots that developed were excised from calli and transferred to a root inducing solid MS medium containing 0.5 mg/L of naphtaleneacetic acid (NAA), 100 mg/L ticarcillin, 3 mg/L potassium clavulanate and 25 mg/L hygromycin. After a 1 to 2 week period of root induction, the plantlets were transplanted to soil.

### EXAMPLES

### Example 1

### Identification of the Effect of Cl⁻ Ions on Transformation Efficiency

This experiment showed that plants germinated in a plant growth medium supplemented with a chloride-containing compound exhibited a higher frequency of homologous recombination when compared to plants grown on a control medium. Moreover, the progeny of these plants also had higher spontaneous levels of HRF.

Exposure of Arabidopsis plants to 25-100 mM NaCl resulted in 2-4-fold increase in recombination frequency as shown in Table 1.

TABLE 1

| NaCl | RR, 10⁻⁸ | Fold Increase |
|---|---|---|
| 0 mM | 7.50 ± 0.01 | 1 |
| 25 mM | 12.29 ± 0.18 | 1.64 |
| 75 mM | 22.52 ± 0.34 | 3 |
| 100 mM | 29.57 ± 0.82 | 3.93 |

Tissues were prepared from A. thaliana plants that were germinated and grown for a period of 3 weeks on a medium containing 0, 25, 75, and 100 mM of NaCl. The recombination rate (RR) was calculated by scoring the HRF in separate groups of 3-week-old plants and then relating these numbers to the total number of genomes present in the plants. Each of the calculated values represents the mean value for the RR. Statistical values are indicated in Table 1, Student's test, α=0.05; for RR t=2.78 and P<0.001. The “Fold Increase” values shown in Table 1 were calculated by relating the data from plants grown on 25, 75 and 100 mM of NaCL to the data from plants grown in the absence of NaCl.

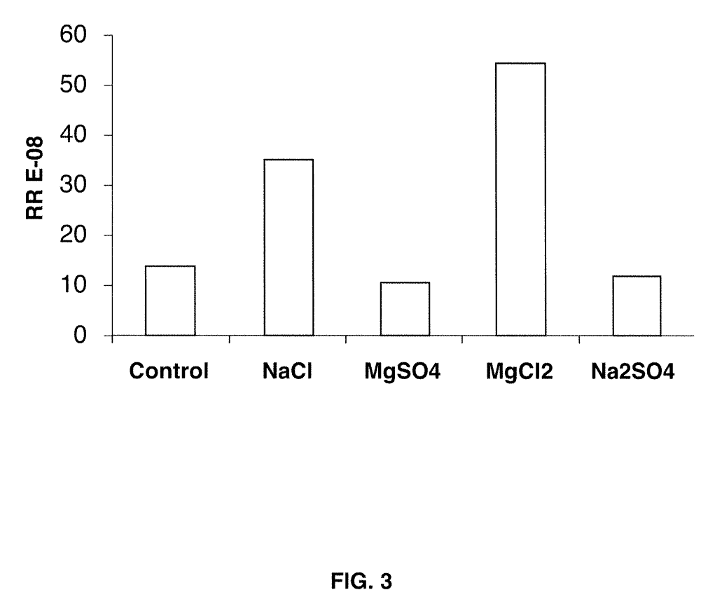

In order to identify which ion had the most significant effect on the RR, several compounds which had different ion combinations were tested: NaCl, Na₂SO₄, MgCl₂ and MgSO₄, and their effects on the HRF and in turn, RR were evaluated. Plants were germinated and grown on MS medium supplemented with one of 25 mM NaCl, 12.5 mM of Na₂SO₄, 12.5 mM of MgCl₂, and 12.5 mM of MgSO₄. Control plants were grown on standard MS medium. The homologous recombination frequency was determined via histochemical staining at 21 days post-germination, results are shown in FIG. 3. The data shown is the RR as an average of two independent experiments, each having 200 plants per experimental group. It was found that only the NaCl and MgCl₂ ion combinations had significantly positive effects on homologous recombination frequency. Through this series of experiments, it was determined that the Cl⁻ ion was responsible for the increased RR.

### Example 2

### Effect of Cl⁻ Containing Compounds on Plant Growth

In the selection of a particular chloride containing compound for supplementing plant growth medium, the effects of both NaCl and KCl on plant growth were evaluated. It was known in the art at that time that the Na⁺ ion was associated with deleterious effects on plants. The experiments of Example 1 demonstrated that the Cl ion was responsible for the increase observed in homologous recombination. However, given the known issues with the use of Na⁺ ions in plants, a less toxic ion, K⁺, was substituted for the Na⁺ ion.

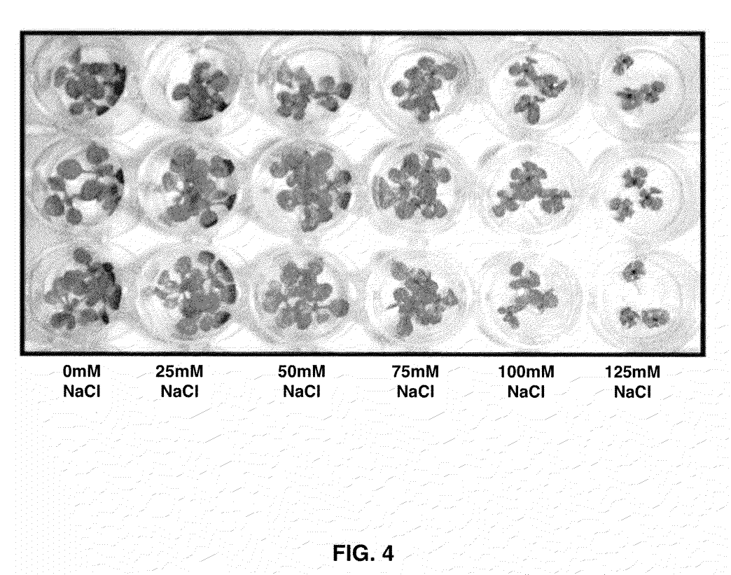

In order to assess the toxicity of Na⁺ ions and K⁺ ions on plant growth, plants were grown on an MS medium supplemented with one of 25, 50, 75 and 100 mM of either NaCl or KCl. The standard MS medium lacked KNO₃. It was determined that plants' exposure to 75 mM of NaCl resulted in about a 20% decrease in plant biomass, whereas exposure to 100 mM of NaCl resulted in about a 50% decrease as shown in FIG. 4. Alternatively, plant exposure to 100 mM of KCl appeared to have little or no effect on plant phenotype, as no decrease in plant biomass was observed. Consequently, KCl was selected for use in further experiments. The K⁺ ion was also selected based on data that illustrated plants cultured on a medium in the absence of a K⁺ ion resulted in lower homologous recombination frequencies on comparison to plants cultured on a MS medium containing a substantial amount of K⁺, specifically 18.8 mM KNO₃ and 1.25 mM KH₂PO₄, totaling in 20.05 mM of K⁺.

### Example 3

### Analysis of Homologous Recombination in Arabidopsis

The effects of KCl on plant transformation were measured using transgenic plants germinated from Arabidopsis line #11 obtained from Prof. Hohn, Friedrich Miescher Institute, Basel, Switzerland. Plants were germinated and grown on a solid or modified MS basal medium in presence of varying quantities of KCl. In order to establish KCl as the single source of potassium in all the modified media, KNO₃, normally present in MS media was omitted. Additionally, potassium dihydrogenphosphate (KH₂PO₄) was replaced with ammonium dihydrogenphosphate (NH₄▪H₂PO₄). In order to compensate for the loss of nitrates from the substituted and deleted components, the concentration of ammonium nitrate (NH₄NO₃) was increased by 18.8 mM to a concentration of 39.4 mM. The control medium composition, a solid MS medium, was not changed.

The frequency of homologous recombination was measured in approximately 200 Arabidopsis plants in each experimental group, germinated and grown on a solid control medium or on a modified solid media containing one of 18.8 (1×), 47 (2.5×) and 94 (5×) mM of KCl, for a period of about 3 weeks. The ‘1×’ stands for the concentration of KNO₃ present in standard MS medium. The experiments were performed in duplicate.

The media compositions used to determine the effects of KCl on recombination frequency and transformation efficiency in N. tabacum are listed below in Table 2.

TABLE 2

| | | Experimental media compositions, | | | |
|---|---|---|---|---|---|
| | | all final concentrations listed in mM | | | |
| MS macro, mM | | Control | KCl, 1x | KCl, 2.5x | KCl, 5x |
| NH₄NO₃ | 20.6 | 39.4 | 39.4 | 39.4 | 39.4 |
| KNO₃ | 18.8 | — | — | — | — |
| CaCl₂ | 3 | 3 | 3 | 3 | 3 |
| MgSO₄ | 1.5 | 1.5 | 1.5 | 1.5 | 1.5 |
| KH₂PO₄ | 1.25 | 1.25 | 1.25 | 1.25 | 1.25 |
| K₂SO₄ | — | 9.4 | 9.4 | 9.4 | 9.4 |
| KCl | — | — | 18.8 | 47 | 94 |

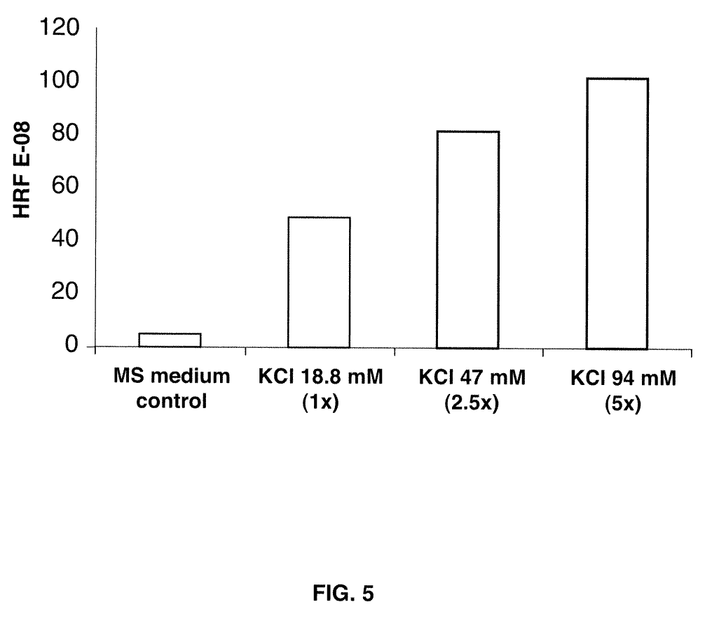

The homologous recombination frequency was measured using histochemical staining for each of the plants grown on the control and modified medium compositions as shown in FIG. 5. Arabidopsis plants that were grown on the modified growth media having 18.8 mM of KCl resulted in a 9.3-fold increase in homologous recombination when compared to plants grown on control MS medium. Similarly, modified growth media having 47 and 94 mM KCl respectively, exhibited 15.4-fold and 19.2-fold increases in homologous recombination respectively, when compared to the plants grown on the control MS medium (Student's t-test, α=0.05). Analyses of the frequency of homologous recombination indicated a strong positive correlation between quantity of KCl present in the modified growth medium and the quantified rates of homologous recombination (r=0.92). The results of these experiments demonstrated that presence of KCl in a growth medium significantly increased the frequency of homologous recombination.

### Example 4

### Analysis of the Effects of KCl on Calli Regeneration in Nicotiana Tabacum

The effects of KCl on the occurrence calli regeneration in N. tabacum plants were evaluated. Calli were regenerated under selective conditions utilizing a selection marker of hygromycin, 25 mg/L.

N. tabacum plants were grown on liquid MS media supplemented with 47 and 94 mM of KCl as shown in Table 3. Plants grown in presence of 47 and 94 mM KCl in liquid medium were used for transformation with luciferase containing a T-DNA construct. Calli were regenerated under selective conditions (hygromycin, 25 mg/L).

TABLE 3

| | | | | | | Integration |
|---|---|---|---|---|---|---|
| | | | | | | frequency |
| | | Calli regenerated and transplanted | | | | LUC |
| | | LUC expression test | | | | ositive |
| | leaves | LUC | LUC | | died | plants/leaf |
| KCl | transformed | positive | negative | total | on soil | transformed |
| 0 mM | 20 | 18 | 5 | 23 | 0 | 0.9 |
| 47 mM | 20 | 106 | 32 | 138 | 0 | 5.3 |
| 94 mM | 20 | 144 | 41 | 181 | 3 | 7.2 |

Media containing 47 and 94 mM of KCl increased the number of calli regenerated by factor of 5.9-fold when compared to the control medium as shown in FIG. 5.

### Example 5

### Analysis of the Effects of KCl on the Frequency of Stable T-DNA Integrations in N. Tabacum

The effects of KCl on the occurrence of stable plant transformation events in N. tabacum plants were evaluated. These experiments identified plants where the DNA of interest had stably integrated into the plant genome.

N. tabacum plants were grown on liquid MS media supplemented with KCl as shown in Table 2. The newly appeared plantlets regenerated from the calli of Example 4 showing evidence of root formation, were excised from the calli and transferred to soil.

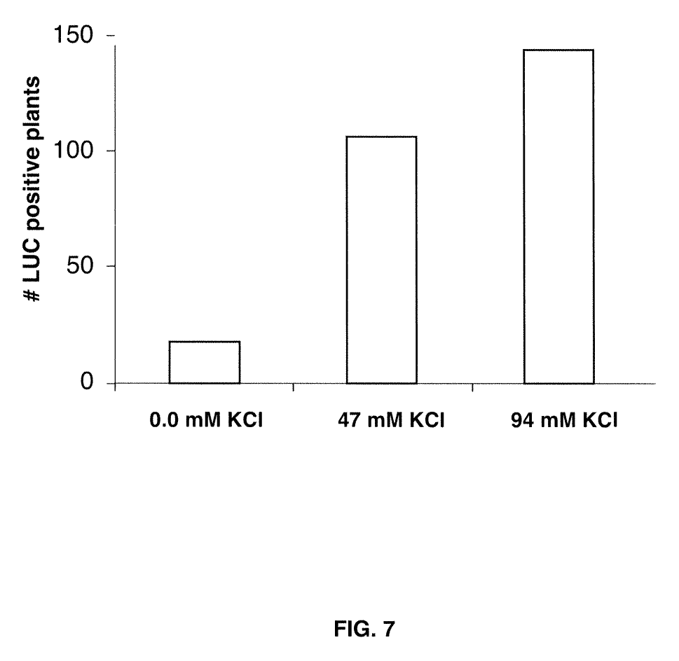

Plantlets were sprayed with luciferine, and the total number of luciferase-positive plantlets was scored. This allowed the calculation of the transformation frequency, as the number of plants expressing LUC gene to the number of transformed leaves, shown in FIG. 7.

The number of stable transformants re-generated from plants grown on KCl supplemented media and control media, as detailed in Example 4, were compared. The comparison of the numbers of stable transformants formed on the media containing 47 and 94 mM KCl and the control media showed a 5.9- and 8.0-fold difference, respectively (FIG. 7).

The above-described embodiments have been provided as examples, for clarity in understanding the invention. A person of skill in the art will recognize that alterations, modifications and variations may be effected to the embodiments described above while remaining within the scope of the invention as defined by the claims appended hereto.

The invention claimed is:

1. A plant culture medium composition for modulating plant transformation events, the composition comprising:
a plant culture medium; and
an effective amount of at least one compound having a chloride component intermixed thereinto.

2. The composition according to claim 1, wherein a baseline level for plant transformation events is provided by culturing at least one of a plurality of plant cells, at least one plant, and plant tissue on said plant culture medium.

3. The composition according to claim 1, wherein said composition further includes additional compounds for increasing the frequency of plant transformation events.

4. The composition according to claim 3, wherein said additional compounds are selected from the group comprising at least one of a rare earth element-containing compound, a nitrate-containing compound, and combinations thereof.

5. The composition according to claim 1, wherein a baseline level for plant transformation events is provided by culturing at least one plant on said plant culture medium.

6. The composition according to claim 1, wherein said at least one chloride-containing compound is selected from the group comprising NaCl, MgCl₂, and KCl.

7. The composition according to claim 1, wherein said at least one chloride-containing compound is KCl.

8. The composition according to claim 1, wherein said at least one chloride-containing compound is KCl in an amount of at least 47 mM.

9. The composition according to claim 1, wherein said at least one chloride-containing compound is KCl in an amount greater than at least 18.8 mM.

10. The composition according to claim 2, wherein on comparison to said baseline level for plant transformation events, said composition increases the number of plant transformation events.

11. The composition according to claim 2, wherein on comparison to said baseline level for plant transformation events, said composition increases the number of plant transformation events by at least one of 2-fold, 3-fold, 4-fold, 5-fold, and 10-fold.

12. The composition according to claim 2, wherein said plurality of plant cells is selected from the group comprising immature embryos, scutellar tissue, suspension cell cultures, immature inflorescence, shoot apical meristem, nodal explants, callus tissue, hypocotyl tissue, cotyledons, roots, and leaves.

13. The composition according to claim 1, wherein a plant cell grown on said plant culture medium composition is selected from the group comprising immature embryos, scutellar tissue, suspension cell cultures, immature inflorescence, shoot apical meristem, nodal explants, callus tissue, hypocotyl tissue, cotyledons, roots, and leaves.

14. The composition according to claim 1, wherein a plant grown on said plant culture medium composition is selected from the group comprising monocots and dicots.

15. The composition according to claim 1, wherein a plant grown on said plant culture medium composition is selected from the group comprising Arabidopsis sp., Nicotiana tabacum, Brassica spp., Solanum lycopersicum, Solanum tuberosum, Zea mays, Triticum spp. Oryza sativa, Papevar spp. and x Triticosecale.

16. The composition according to claim 1, wherein said plant culture medium is selected from the group comprising isolation media, pre-culture media, induction media, inoculation media, delay media, selection media, and regeneration media.

17. A method for modulating the frequency of plant transformation events, the method comprising the steps of:
a) providing the plant culture medium composition of claim 1;
b) contacting at least one plant with the plant culture medium composition;
c) transforming at least one cell from said at least one plant with a nucleic acid of interest;
d) detecting the presence of at least one transformation event, and
e) quantifying said transformation event;
f) comparing the frequency of said quantified transformation events with a suitable control;
wherein changes in the quantified transformations events compared to the control are indicative of a change in the frequency of plant transformation events.

18. The method according to claim 17, wherein said at least one chloride-containing compound is selected from the group comprising NaCl, MgCl₂, and KCl.

19. The method according to claim 17, wherein the composition further includes at least one of a rare earth element-containing compound, a nitrate-containing compound, and combinations thereof.

20. The method according to claim 17, wherein said at least one chloride-containing compound is KCl.

21. The method according to claim 17, wherein said at least one chloride-containing compound is KCl is provided in an amount of at least 47 mM.

22. The method according to claim 17, wherein said at least one chloride-containing compound is KCl in an amount greater than at least 18.8 mM.

23. The method according to claim 17, wherein said suitable control is selected from the group comprising a stored dataset of results generated from studies of the presence and expression transformation events in one or more population(s) of plants grown on said plant culture medium, a stored dataset of results generated from studies of the presence and expression of transformation events in one or more population(s) of plant cells grown on said plant culture medium, a stored dataset of results generated from studies of the presence and expression transformation events in one or more population(s) of plant tissue grown on said plant culture medium and combinations thereof.

24. The method according to claim 17, wherein said suitable control is a baseline level for plant transformation events and is provided by culturing at least one of a plurality of plant cells, at least one plant, and plant tissue on said plant culture medium.

25. The method according to claim 17, wherein said changes in the quantified transformations compared to the control, are an increase in the frequency of plant transformation events.

26. The method according to claim 17, wherein said changes in the quantified transformations compared to the control are an increase in the frequency of plant transformation events by at least one of 2-fold, 3-fold, 4-fold, 5-fold, and 10-fold.

27. The method according to claim 17, wherein said at least one plant is selected from the group comprising monocots and dicots.

28. The method according to claim 17, wherein said at least one plant is selected from the group consisting Arabidopsis sp., Nicotiana tabacum, Brassica spp., Solanum lycopersicum, Solanum tuberosum, Zea mays, Triticum spp., Oryza sativa, Papevar spp. and x Triticosecale.

29. The method according to claim 17, wherein said plant culture medium is selecting from the group comprising isolation media, pre-culture media, induction media, inoculation media, delay media, selection media, and regeneration media.

30. A method for transforming a plant cell, the method comprising the steps of:
a) providing the plant culture medium composition of claim 1;
b) contacting a plurality of plant cells with the plant culture medium composition;
c) transforming said plurality of plant cells with a nucleic acid of interest;
d) detecting the presence of at least one transformation event; and
e) regenerating at least one transformed plant from at least one transformed plant cell.

31. The method according to claim 30, wherein said transformed plant cells produces a transgenic plant.

32. The method according to claim 30, wherein said at least one chloride-containing compound is selected from the group comprising: NaCl, MgCl₂, and KCl.

33. The method according to claim 30, wherein the composition further includes at least one of a rare earth element-containing compound, a nitrate-containing compound, and combinations thereof.

34. The method according to claim 30, wherein said at least one chloride-containing compound is KCl.

35. The method according to claim 30, wherein said at least one chloride-containing compound is KCl provided in an amount of at least 47 mM.

36. The method according to claim 30, wherein said at least one chloride-containing compound is KCl in an amount greater than at least 18.8 mM.

37. The method according to claim 30, wherein said plurality of plant cells is selected from the group comprising immature embryos, scutellar tissue, suspension cell cultures, immature inflorescence, shoot apical meristem, nodal explants, callus tissue, hypocotyl tissue, cotyledons, roots, and leaves.

38. The method according to claim 30, wherein said plant culture media is selecting from the group comprising isolation media, pre-culture media, induction media, inoculation media, delay media, selection media, and regeneration media.

39. Use of an effective amount of a chloride-containing compound intermixed into a plant culture medium to modulate plant transformation.

40. The use according to claim 39, wherein said chloride-containing compound is selected from the group comprising NaCl, MgCl₂, and KCl.

41. The use according to claim 39, wherein said plant culture medium further includes at least one of a rare earth element-containing compound, a nitrate-containing compound, and combinations thereof.

42. The use according to claim 39, wherein said chloride-containing compound is KCl.

43. The use according to claim 39, wherein said chloride-containing compound is KCl provided in an amount of at least 47 mM.

44. The use according to claim 39, wherein said chloride-containing compound is KCl in an amount greater than at least 18.8 mM.

45. The use according to claim 39, wherein said plant culture medium is selecting from the group comprising isolation media, pre-culture media, induction media, inoculation media, delay media, selection media, and regeneration media.
